(19) United States
(12) Patent Application Publication
(10) Pub. No.: US 2003/0187370 A1
(43) Pub. Date: Oct. 2, 2003

(54) Uterine contraction sensing system and method

(76) Inventor: Roy K. Kodama, Thousand Oaks, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/113,890

(22) Filed: Mar. 27, 2002

(51) Int. Cl. A61B 5/103

(52) U.S. Cl. 600/591

(57) ABSTRACT

A system and method for enabling the sensing of changes in pressure in response to uterine muscle tone changes relating to contractions, and for enabling the evaluation of contractions based thereon. The system, in an embodiment thereof, enables ease of use and enhanced patient comfort. It also enables maximized sensitivity and minimized system loading retention pressure. Further, it enables a portion of the system to comfortably and efficiently project below the non-compressed abdominal surface, and provides electrical isolation of the system elements.

### BACKGROUND OF THE INVENTION

1. Field of the Invention

The present invention relates generally to improvements in sensing uterine contractions during labor. In particular, it relates to improved systems and methods for sensing the frequency and duration of contractions, and for estimating the relative intensity thereof.

2. Description of the Related Art

The systems and methods of the present invention are particularly useful for sensing changes in pressure in response to uterine muscle tone changes relating to contractions, and for enabling a fetal monitor to be connected thereto for evaluation of contractions.

During a contraction, the muscle tone of the uterus increases. This increase in muscle tone applies pressure to the abdomen. The abdomen hardens and the shape changes due in part to the muscles surrounding the anterior ligament of the uterus pulling the abdomen forward.

A variety of systems and methods have been developed over the years for estimating the force of uterine contractions during labor, to enable evaluation thereof. Such systems are known generally as tocodynamometers, which sense in relative terms the increase and decrease in abdominal pressure relating to uterine contractions. Such systems typically include a cantilevered beam as a sensor, and a button as a force collector, which is connected to the sensor by a link pin. The output from the system is sent to a fetal monitor, which displays the relative pressure in the form of a digital reading and strip chart recording. The clinician, in reviewing the chart recording, can observe the progress of labor in terms of contraction frequency and duration, and can obtain a rough indication of the relative intensity of the contractions.

However, there have been problems associated with such systems. They are relatively large and bulky, require relatively large devices to hold them in place, and are expensive. Further, they are relatively difficult to use, require relatively high belt tension which is uncomfortable, and tend to migrate and require repositioning.

Therefore, the present invention provides improved systems and methods for enabling the efficient and effective sensing of the frequency and duration of uterine contractions during labor, and the estimating of the relative intensity thereof. It also enhances ease of use and patient comfort. Further, the sensitivity of the system is maximized. The improved systems and methods also enable unique leveraging thereof, for loading onto the abdomen with the exertion of substantially minimal retention pressure. Also, the invention further enables support of the system so as to project into the abdomen wall to efficiently reside below the non-compressed abdominal surface. The invention further enables the system to provide electrical isolation of the patient, to prevent the flow of current thereto. The inventions disclosed herein satisfy these and other needs.

### SUMMARY OF THE INVENTION

The present invention, in general, provides a new and improved system and method for sensing the frequency and duration of uterine contractions during labor and estimating the relative intensity thereof. It enables the sensing of changes in pressure in response to uterine muscle tone changes relating to contractions, and the evaluation of contractions upon connection thereof to a fetal monitor. It is small and lightweight, enhancing ease of use and patient comfort. It also minimizes the size of operational elements and/or portions of an operational element, so as to maximize the sensitivity of the system. The invention further leverages the system, so as to enable the loading thereof onto the abdomen with the exertion of substantially minimal retention pressure thereon. It further minimizes migration thereof on the patient, to increase patient comfort and prevent the need for frequent repositioning thereof. Also, it enables the system to be supported such that a portion of the system projects into the abdomen wall to reside below the non-compressed surface of the abdomen. The invention further provides electrical isolation, to prevent current from flowing to the patient.

More particularly, for example, in an embodiment of the present invention, a system is provided for sensing the frequency and duration of uterine contractions during labor and estimating the relative intensity thereof. The system senses changes in pressure in response to uterine muscle tone changes relating to contractions, and enables a fetal monitor to be connected thereto for evaluation of contractions. The system includes a sensing element, for sensing changes in pressure responsive to uterine muscle tone changes, positionable against the exterior abdominal wall proximate the uterine muscle, and connectable to a fetal monitor for evaluation of contractions during labor. It also includes a supporting element, for supporting the sensing element so as to bear against the exterior abdominal wall proximate the uterine muscle.

The above objects and advantages of the present invention, as well as others, are described in greater detail in the following description, when taken in conjunction with the accompanying drawings of illustrative embodiments.

### BRIEF DESCRIPTION OF THE DRAWINGS

FIG. 1 is an exploded top perspective view of a system in accordance with an embodiment of the present invention.

FIG. 2 is a bottom perspective view of a sensing member and a supporting member pursuant to an embodiment of the invention.

FIG. 3 is a bottom partly-fragmentary plan view of the system, in an embodiment of the invention.

FIG. 4 is a top plan view of a sensing element and a supporting element in the system, in the practice of the invention.

FIG. 5 is an elevational partly-sectional view of a sensing element and a supporting element in the system, pursuant to the invention.

FIG. 6 is an elevational partly-sectional view of a sensing element in the system, in another embodiment of the invention.

FIG. 7 is an elevational partly-fragmentary view of the system including a ridge area in the supporting element, pursuant to the invention.

FIG. 8 is an elevational partly-fragmentary view of the system including a counterbore area in the supporting element, in the practice of the invention.

### DETAILED DESCRIPTION OF THE PREFERRED EMBODIMENTS

The present invention is directed to an improved system and method for sensing the frequency and duration of uterine contractions during labor, and estimating the relative intensity thereof, in an efficient and effective manner. The invention enables the sensing of changes in pressure in response to uterine muscle tone changes relating to contractions, and enables a fetal monitor to be connected thereto for evaluation of contractions. The system of the invention is small and lightweight, enhancing ease of use and patient comfort. The relative size is minimized of operational elements and/or portions of an operational element, to maximize the sensitivity of the system. The present invention is further directed to uniquely leveraging the system, so as to enable the loading thereof onto the abdomen with substantially minimal retention pressure exerted thereon. Such leveraging, even with minimal retention pressure, and the small size and light weight of the system, minimizes migration thereof on the patient, which would otherwise increase patient discomfort and require frequent repositioning thereof. Additionally, the present invention enables the system to be supported such that a portion of the system projects into the abdomen wall so as to reside below the non-compressed surface of the abdomen. Further, the invention provides electrical isolation, to prevent current from flowing to the patient. The preferred embodiments of the improved system and method are illustrated and described herein by way of example only and not by way of limitation.

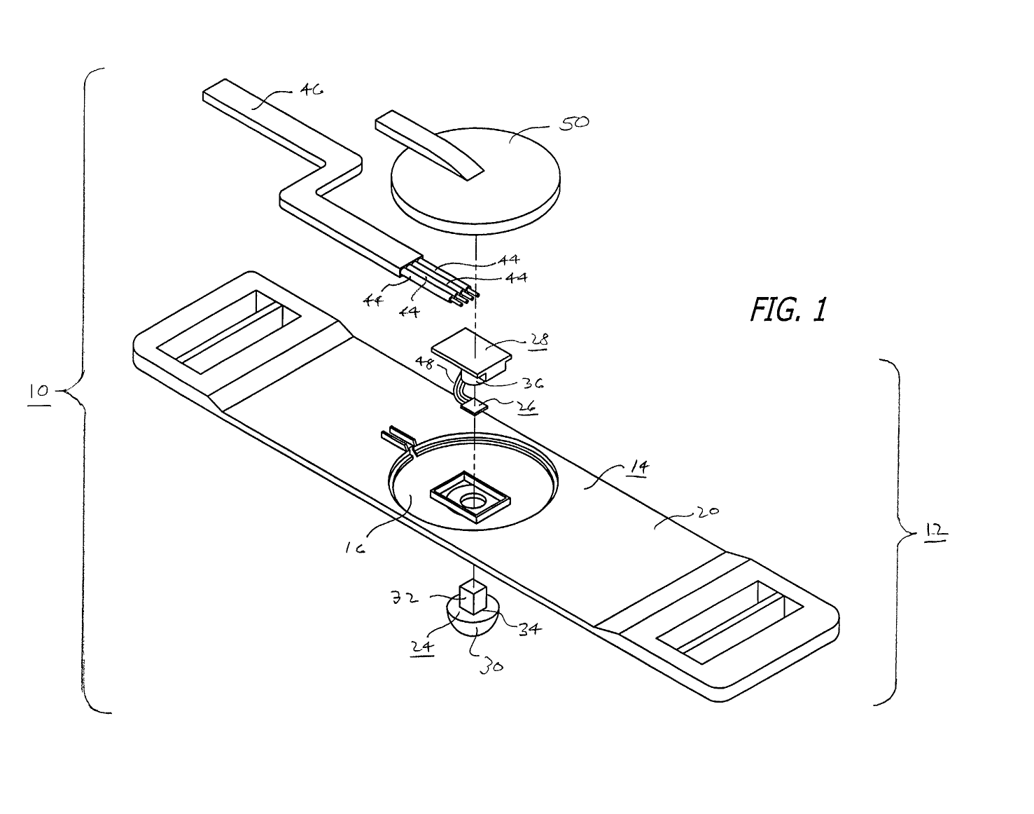

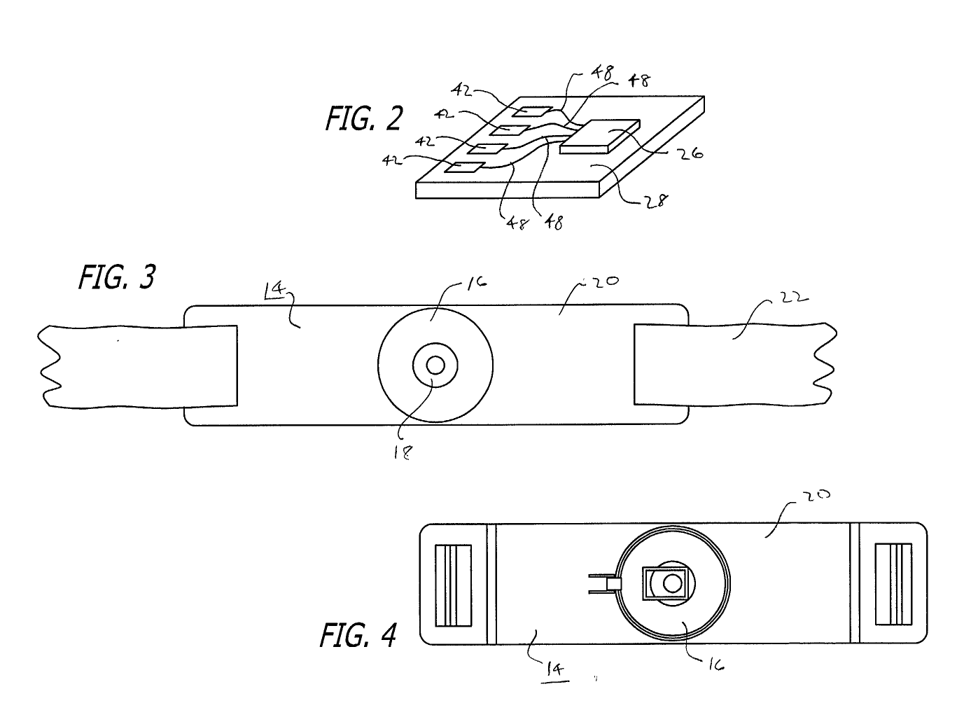

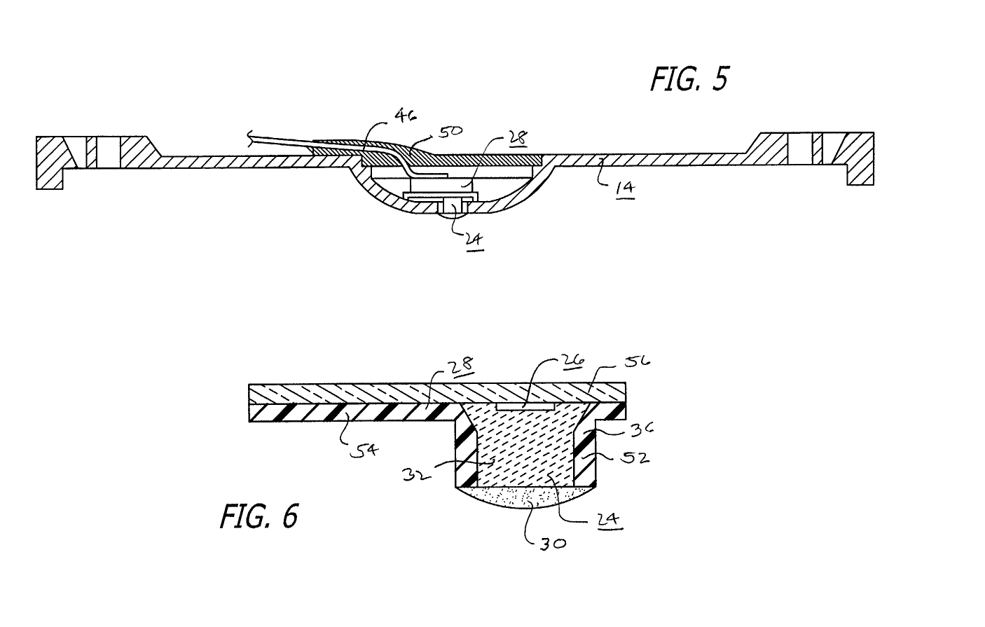

In the drawings, wherein like reference numerals denote like or corresponding parts throughout the drawing figures, and particularly in the preferred embodiments in accordance with the invention as shown in FIGS. 1-8, for example, a system 10 is provided for enabling the sensing and monitoring of uterine contractions.

Referring to FIGS. 1-8, in a preferred embodiment pursuant to the present invention, for example, the system 10 includes a sensing element 12, for sensing changes in pressure responsive to uterine muscle tone changes. The sensing element 12 is positionable against the exterior abdominal wall proximate the uterine muscle. It is connectable to a fetal monitor for evaluation of contractions during labor. The sensing element 12 is generally small and lightweight. The sensing element 12 is also able to be actuated by an excitation voltage from a fetal monitor, and to send a signal voltage back to the fetal monitor responsive to changes in pressure.

The system 10 also includes a supporting element 14, for supporting the sensing element 12 so as to bear against the exterior abdominal wall proximate the uterine muscle. The supporting element 14 is also generally low profile and lightweight. It is able to support the sensing element 12 such that the sensing element 12 projects into the abdominal wall so as to reside below the non-compressed surface of the abdomen. The supporting element 14 includes a housing which includes a mounting portion 16 for mounting the sensing element 12 therein, is generally semi-spherical in shape, generally in the form of a dome, and includes a recess 18 in the form of a well therein. The housing further includes a generally rectangular-shaped back portion 20 from which the mounting portion 16 projects. The sensing element 12 is able to project from and be moveable in the recess 18 of the supporting element 14.

The system 10 further includes an extending element 22, for extending about the exterior abdominal wall so as to retain the supporting element 14 and the sensing element 12 in position against the exterior abdominal wall proximate the uterine muscle. The extending element 22 for example comprises a belt, which is comprised of a generally elastic material. The generally elastic material may for example constitute an elastic nylon material, or an elastic polyester material. The supporting element 14 also leverages the sensing element 12, so as to enable the extending element 22 to load the sensing element 14 onto the abdomen, with substantially minimal retention pressure exerted by the extending element 22 on the sensing element 12. Further, the supporting element 14 is comprised of a flexible material or a non-flexible material, such that, in conjunction with the extending element 22, the flexible material or the non-flexible material maintains a load on the sensing element 12 during abdominal changes in response to contractions. The flexible material of which the supporting element 14 is comprised is preferably a flexible plastic material, such as polycarbonate, which flexible plastic material may constitute for example a thermoplastic.

The sensing element 12 includes a moving member 24, for moving responsive to uterine muscle tone changes, as by displacing or compressing so as to exert pressure responsive thereto, and a sensing member 26, for sensing movement of the moving member 24, and for enabling a fetal monitor to be connected thereto. It may also include a supporting member 28, for supporting the moving member 24 and the sensing member 26. Alternatively, for example, the supporting element 14 may include a supporting member for supporting the moving member 24. The supporting member may alternatively for example be formed as a supporting portion of the supporting element 14, or for example as a supporting part which is extendable between the sensing element 12 and the supporting element 14.

As illustrated in FIGS. 1-5 and 7-8, in an embodiment in accordance with the invention, the moving member 24 for example comprises a force collector. The force collector 24 for example is generally in the shape of a nipple, and is comprised of a generally soft and compliant material. The generally compliant material of which the force collector 24 is comprised for example may be a generally compliant polymer material, a generally compliant monomer material, or another generally compliant material such as latex. The force collector 24 may alternatively for example be comprised of a generally non-compliant material which includes compliant material therein, such as an oil. The generally compliant material and the generally non-compliant material may comprise a generally soft low durometer material or a generally non-low durometer non-compliant material. The moving member 24 and the supporting member 28 support the sensing member 26 so as to isolate the sensing member 26 from the external environment, to prevent current from flowing to the patient. The supporting element 14 is further able to support the moving member 24 such that the moving member 24 projects into the abdominal wall so as to reside below the non-compressed surface of the abdomen. The moving member 24 includes an outer portion 30, which is comprised for example of an ultraviolet-cured flexible adhesive, and which projects from the supporting element 14, and an inner portion 32, which is comprised for example of a silicone gel, and which contacts the sensing member 26. There is an interface 34 between the outer portion 30 and the inner portion 32 of the moving member 24. The moving member 24 projects from the recess 18 in the supporting element 14 and is moveable therein.

The sensing member 26 comprises the active area of the sensing element 12. It includes resistors, which may comprise for example thin film resistors deposited thereon, which resistors are able to change the resistance with changes in applied pressure. The resistors in the sensing member 26 may comprise a balanced resistor network, which may comprise a silicon semiconductor Wheatstone bridge. The sensing member 26 and the supporting member 28 for example may comprise a pressure transducer. Alternatively for example the sensing member 26 and the supporting portion of the supporting element 14 may comprise a pressure transducer, or for example the sensing member 26 and the supporting part of the supporting element 14 may comprise a pressure transducer. The supporting member 28 may include compensating resistors therein, for providing compensation for the resistors in the sensing member 26.

The moving member 24 is formed such that the ratio of the moving member 24 to the sensing member 26, and/or the ratio of the outer portion 30 of the moving member 24 to the interface 34 between the outer portion 30 and the inner portion 32 of the moving member 24, is substantially minimal, so as to maximize the sensitivity of the system 10.

The ratio of the moving member 24 to the sensing member 26 comprises the moving member-sensing member ratio. The moving member-sensing member ratio may comprise the ratio of the width of the moving member 24 to the width of the sensing member 26. The width of the moving member 24 and the width of the sensing member 26 may comprise the width in any direction thereof. The moving member-sensing member width ratio may be preferably no greater than about three and one-half to one, and may particularly be about two and one-half to one. The moving member-sensing member ratio may alternatively or additionally comprise the ratio of the area of the moving member to the area of the sensing member. The moving member-sensing member area ratio may be preferably no greater than about 8 to 1. For example, the moving member-sensing member area ratio is about 5 to 1 (0.04909 inches/0.01 inches), which is about 5 to 1, where the cross-sectional area of the outer portion 30 of the moving member 24 is (0.25 inches+2)²×3.1416 which equals 0.04909 square inches, and the cross-sectional area of the sensing member 26 is 0.1 inches×0.1 inches which equals 0.01 square inches.

The ratio of the outer portion 30 of the moving member to the interface 34 between the outer portion 30 and the inner portion 32 of the moving member comprises the moving member ratio. The moving member ratio may comprise the ratio of the cross-sectional area of the width of the outer portion 30 of the moving member 24 to the cross sectional area of the interface 34 between the outer portion 30 and the inner portion 32 of the moving member 24. The cross-sectional area of the width of the outer portion 30 of the moving member 24 may comprise the cross-sectional area of the widest width thereof. Preferably, the moving member area ratio may be no greater than about eight to one, and particularly may be about five to one. The moving member ratio in particular may alternatively or additionally comprise the width ratio of the linear width of the outer portion 30 of the moving member 24 to the linear width of the interface 34 between the outer portion 30 and the inner portion 32 of the moving member 24. The linear width of the outer portion 30 of the moving member 24 may comprise the linear widest width thereof. The moving member width ratio may preferably be no greater than about 3.5 to 1, and may particularly be about 2.5 to 1. The moving member width ratio, for example, is about 2.27 to 1 (0.25 inches/0.110 inches), which is less than about 2.5 to 1, where the diameter of the outer portion 30 of the moving member 24 is 0.25 inches, and the width in any direction of the interface 34 between the outer portion 30 and the inner portion 32 of the moving member 24 is 0.110 inches.

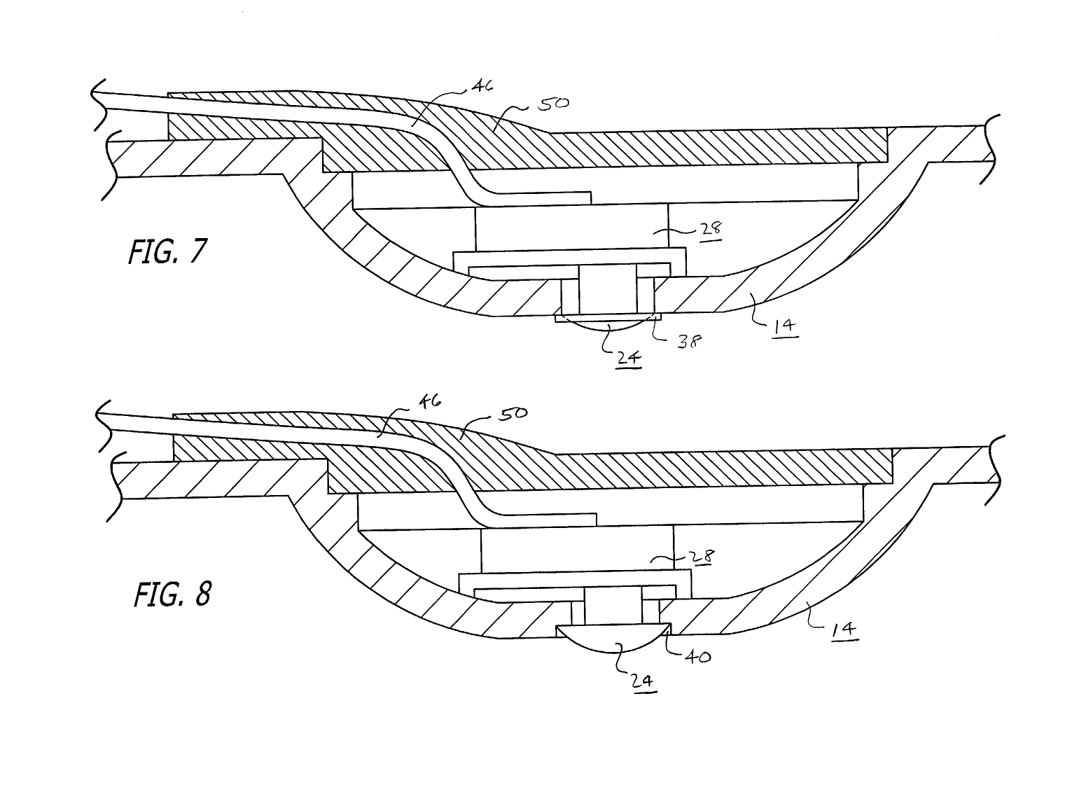

The supporting member 28 supports the sensing member 26 thereon, and may for example include a gel cup 36, comprised of generally molded plastic material, which is filled with a non-conductive silicone gel, a bonding element, for bonding the sensing member 26 to the gel cup 36, and a ceramic chip substrate. The supporting member 28 may alternatively for example comprise a gel cup, comprised of generally molded plastic material, and a ceramic chip substrate, or for example may comprise a ceramic chip substrate. The ceramic chip substrate in the supporting member 28 may include compensating resistors therein, for providing compensation for the resistors in the sensing member 26. The moving member 24 is formed of a material which is able to be dispensed and formed relative to the gel cup 36. The gel cup 36 includes an adhering portion, for enabling the material of which the moving member 24 is formed to adhere thereto. The supporting element 14 may include an extending portion, comprising a ridge area 38, as seen in FIG. 7, or a counterbore area 40, as shown in FIG. 8, each of which comprises a detail such as a meniscus, and which is formed relative to the adhering portion, for inhibiting the material of which the moving member 24 is formed from flowing beyond the adhering portion. The ridge area 38 in FIG. 7, for example, may be about 0.005 inches high by 0.010 inches wide. The extending portion enables controlled adhesion of the material to the supporting member 28 and the supporting element 14, so as to prevent the material from affecting the sensitivity of the system. The sensitivity of the active area in the sensing member 26 is affected by the extent of the moving member which is supported thereon which extends beyond the width of the sensing member 26 and is outside the active area thereof. The extending portion reduces the relative surface tension of the supporting element 14 beyond the extending portion for the nipple material of the moving member 24 thereat, such that the nipple material resists flowing therebeyond.

Further in the system 10, the supporting member 28 includes a plurality of contact pads 42, for receiving a plurality of connectors 44 in a cable 46 for a fetal monitor. A plurality of wires 48 connect the sensing member 26 to the contact pads 42. In the event of the application of a voltage or current exceeding a limit, the wires 48 connecting the sensing member 26 to the contact pads 40 will fail substantially instantaneously, preventing current from flowing to the patient. The system 10 also includes a cover 50, for covering the supporting member 28, the sensing member 26, and a portion of the fetal monitor cable 46.

In an embodiment of the invention as shown in FIG. 6, the inner portion 32 of the moving member 24 comprises a silicone gel, and the outer portion 30 of the moving member 24 comprises an ultraviolet-cured flexible adhesive. The supporting member 28 supports the sensing member 26 and the inner portion 32 of the moving member 24 therein, and supports the outer portion 30 of the moving member 24 thereon. The supporting member 28 comprises a gel cup 36 which includes a wall portion 52, an extending base portion 54, and a ceramic chip substrate 56 secured to the extending base portion 54. The sensing member 26 comprises a piezo-resistive pressure sensor. The ceramic chip substrate 56 may include compensating resistors therein, for providing compensation for the resistors in the sensing member 26. The inner portion 32 of the moving member 24 is formed in the supporting member 28 so as to extend over and about the piezo-resistive pressure sensor 26.

Referring to FIGS. 1-5 and 7-8, in a method for the use of a preferred embodiment in accordance with the present invention, for example, the system 10 enables the sensing of the frequency and duration of uterine contractions during labor, and the estimating of the relative intensity thereof. The system 10 is placed on the patient's abdomen, for example in the fundal area thereof, and is retained in position by the belt 22, comprised of generally elastic material, which may comprise elastic nylon material or elastic polyester material. The belt 22 presses the supporting element 14 and the sensing element 12 such that the moving member 24 of the sensing element 12 presses firmly against the exterior abdominal wall. The sensing element 12 is connectable to a fetal monitor for evaluation of contractions, senses changes in pressure responsive to uterine muscle tone changes, is actuated by an excitation voltage from the fetal monitor, and sends a signal voltage back to the fetal monitor responsive to changes in pressure.

The generally small and lightweight sensing element 12 senses changes in pressure in response to uterine muscle tone changes relating to contractions.

The moving member 24 of the sensing element 12, which comprises a generally nipple-shaped force collector, moves by displacing or compressing so as to exert pressure responsive to uterine muscle tone changes. Movement of the moving member 24 is sensed in the pressure transducer comprised of the sensing member 26 and the supporting member 28, and the resistance in the sensing member 26 changes, thereby changing the signal voltage sent back to the fetal monitor. The pressure transducer may for example alternatively be comprised of the sensing member 26 and the supporting portion of the supporting element 14, or the sensing member 26 and the supporting part of the supporting element 14.

The moving member 24 may be formed for example of a material which is adapted to be dispensed and formed relative to the gel cup 36, for example by dispensing the material of which it is comprised from a syringe in drops, then rapidly curing the material with ultraviolet light. The moving member 24 may further be formed such that the ratio of the moving member 24 to the sensing member 26, constituting the moving member-sensing member ratio, is substantially minimal. In particular, the moving member 24 may be formed such that the ratio of the width, in any direction, of the moving member 24 to the width, in any direction, of the sensing member 26 is substantially minimal, for example no greater than about three and one-half to one, such as about two and one-half to one.

The moving member 24 may also be formed such that the ratio of the outer portion 30 of the moving member 24 to the interface 34 between the outer portion 30 and the inner portion 32 of the moving member 24, constituting the moving member ratio, is substantially minimal. Particularly, the moving member 24 may be formed such that the ratio of the area of the outer portion 30 of the moving member 24 to the area of the interface 34 between the outer portion 30 and the inner portion 32 of the moving member 24 is substantially minimal, for example no greater than about eight to one, such as about four to one. Specifically, the moving member 24 may be formed such that the area ratio of the cross-sectional area of the outer portion 30 of the moving member 24 to the cross-sectional area of the interface 34 is substantially minimal. Alternatively, or additionally, the moving member 24 may be formed such that the width ratio of the linear widest width of the outer portion 30 of the moving member 24 to the linear width of the interface 34 is substantially minimal.

The moving member 24 may be comprised of material which adheres to the adhering portion of the gel cup 36, while the extending portion of the supporting element 14 inhibits the flow thereof substantially beyond the adhering portion. The generally soft and compliant material of which the moving member 24 is comprised, which may comprise a low durometer compliant polymer or monomer, or a generally low durometer non-compliant material, moves as by displacing or compressing so as to exert pressure to project the outer portion 30 thereof, and such that the inner portion 32 thereof contacts the sensing member 26. The moving member 24 may for example alternatively be comprised of generally non-compliant non-low durometerial material, which may include compliant material therein, for enabling such movement thereof. The moving member 24 is supported in the gel cup 36 of the supporting member 28, in the recess in the generally semi-spherically shaped portion of the supporting element 14. The moving member 24 may alternatively for example be supported on the ceramic resistor chip substrate of the supporting member 28.

The supporting element 14 supports the sensing element 12 so that the sensing element 12 is positionable to bear against the exterior abdominal wall proximate the uterine muscle. The supporting element 14 supports the moving member 24 of the sensing member 26 of the sensing element 12 so that the sensing element 12 projects into the abdominal wall to reside below the non-compressed surface of the abdomen. Alternatively, for example, the supporting element 14 supports the moving member 24 in the supporting member therein, which may constitute a supporting portion of the supporting element 14, or a supporting part extending between the sensing element 12 and the supporting element 14.

The flexible material of which the supporting element 14 is comprised, which may comprise a flexible plastic material, in conjunction with the extending element 22, maintains the load on the sensing element 12 during abdominal changes relating to contractions, including breathing and patient movement. The supporting element 14 alternatively for example may be comprised of non-flexible material, and maintains the load on the sensing element 12 thereby. The moving member 24 and the supporting member 28 also support the sensing element 12 so that the sensing element 12 is isolated from the external environment, preventing current from flowing to the patient. The supporting element 14 further leverages the sensing element 12, such that the extending element 22 loads the sensing element 12 onto the abdomen while exerting substantially minimal retention pressure on the sensing element 12.

The resistors, in the active area of the sensing member 26 which may comprise a balanced resistor network comprising a silicon semi-conductor Wheatstone bridge, change resistance with changes in applied pressure, to sense movement of the moving member 24 therein. The housing of the supporting element 14 supports the sensing member 26 in the well of the dome therein. The wires 48 connecting the sensing member 26 to the contact pads 42 fail substantially instantaneously in the event of the application of a voltage or current exceeding a limit. The extending element 22, such as the belt comprised of generally elastic nylon or polyester material, retains the sensing element 12 and the supporting element 14 in position against the exterior abdominal wall proximate the uterine muscle.

As illustrated in FIG. 6, in a method for the use of another embodiment of the invention, for example, the inner portion 32 of the moving member 24 is formed in the wall portion 52 and on the ceramic compensating resistor chip substrate 56 of the supporting member 28, and over and about the piezo-resistive pressure sensor of the sensing member 26.

In accordance with the present invention, the particular embodiments set forth above for the system 10 are capable of providing sensing of the frequency and duration of uterine contractions during labor. However, other forms of the system 10 may be utilized with the present invention without departing from the spirit and scope of the invention. Based on the present disclosure, other constructions and applications are known to one skilled in the art.

In view of the above, it is apparent that the system and method of the preferred embodiments of the present invention enhances substantially the effectiveness of sensing uterine contractions and estimating the relative intensity thereof. Further, the system and method enable the sensing of changes in pressure responsive to uterine muscle tone changes relating to contractions. The system and method also enable connection of a fetal monitor thereto, for evaluation of contractions. The system is small and lightweight, for enhanced ease of use and patient comfort. For maximized system sensitivity, the relative size is minimized of the operational elements and/or portions of an operational element. The system and method further enable the leveraging thereof so as to provide loading onto the abdomen while exerting substantially minimal retention pressure thereon. Migration of the system on the patient is minimized by such leveraging, so as to inhibit patient discomfort and frequent repositioning thereof, even with such system small size, light weight, and minimal retention pressure. Also, the system and method enable support thereof to enable a portion to project into the abdominal wall so as to reside below the non-compressed surface of the abdomen. The system and method further inhibit the flow of current to the patient to provide electrical isolation.

While the present invention has been described in connection with the specific embodiments identified herein, it will be apparent to those skilled in the art that many alternatives, modifications and variations are possible in light of the above description. Accordingly, the invention is intended to embrace all such alternatives, modifications and variations as may fall within the spirit and scope of the invention disclosed herein.

What is claimed is:

1. A system for sensing the frequency and duration of uterine contractions during labor and estimating the relative intensity thereof, adapted to sense changes in pressure in response to uterine muscle tone changes relating to contractions, and to enable a fetal monitor to be connected thereto for evaluation of contractions, comprising:
a sensing element, for sensing changes in pressure responsive to uterine muscle tone changes, adapted to be positioned against the exterior abdominal wall proximate the uterine muscle, and to be connected to a fetal monitor for evaluation of contractions during labor; and
a supporting element, for supporting the sensing element so as to bear against the exterior abdominal wall proximate the uterine muscle.

2. The system of claim 1, wherein the sensing element includes a moving member, for moving by displacing or compressing so as to exert pressure responsive to uterine muscle tone changes, and a sensing member, for sensing movement of the moving member, and for enabling a fetal monitor to be connected thereto.

3. The system of claim 1, wherein the sensing element is generally small and lightweight, and the supporting element is generally low profile and lightweight.

4. The system of claim 1, wherein the sensing element is further adapted to be actuated by an excitation voltage from a fetal monitor, and to send a signal voltage back to the fetal monitor responsive to changes in pressure.

5. The system of claim 1, further comprising an extending element, for extending about the exterior abdominal wall, adapted to retain the supporting element and the sensing element in position against the exterior abdominal wall proximate the uterine muscle.

6. The system of claim 1, wherein the supporting element is adapted to support the sensing element such that the sensing element projects into the abdominal wall so as to reside below the non-compressed surface of the abdomen.

7. The system of claim 2, wherein the moving member comprises a force collector.

8. The system of claim 2, wherein the moving member includes an outer portion adapted to project from the supporting element, an inner portion adapted to contact the sensing member, and an interface between the outer portion and the inner portion.

9. The system of claim 2, wherein the moving member is comprised of material which is generally soft and compliant.

10. The system of claim 2, wherein the sensing element further includes a supporting member, for supporting the moving member and the sensing member.

11. The system of claim 2, wherein the supporting element includes a supporting member for supporting the moving member.

12. The system of claim 2, wherein the moving member and the supporting element are further adapted to support the sensing member so as to isolate the sensing member from the external environment, to prevent current from flowing to the patient.

13. The system of claim 2, wherein the sensing member comprises the active area of the sensing element, and includes resistors adapted to change the resistance with changes in applied pressure.

14. The system of claim 2, wherein the supporting element is adapted to support the moving member such that the moving member projects into the abdominal wall so as to reside below the non-compressed surface of the abdomen.

15. The system of claim 2, wherein the moving member is adapted to be formed such that the ratio of the moving member to the sensing member is substantially minimal.

16. The system of claim 5, wherein the supporting element is adapted to leverage the sensing element, so as to enable the extending element to load the sensing element onto the abdomen, with substantially minimal retention pressure exerted by the extending element on the sensing element.

17. The system of claim 5, wherein the supporting element is comprised of a flexible material, such that, in conjunction with the extending element, the flexible material maintains a load on the sensing element during abdominal changes relating to contractions.

18. The system of claim 5, wherein the supporting element is comprised of a non-flexible material, such that, in conjunction with the extending element, the non-flexible material maintains a load on the sensing element during abdominal changes relating to contractions.

19. The system of claim 5, wherein the extending element comprises a belt.

20. The system of claim 6, wherein the supporting element includes a housing which includes a back plate, and a dome, projecting from the back plate, and wherein the dome has a well therein, and the sensing element is adapted to be mounted in the well of the dome.

21. The system of claim 7, wherein the force collector is generally in the shape of a nipple, and is comprised of a generally compliant material.

22. The system of claim 7, wherein the force collector is generally in the shape of a nipple, and is comprised of a generally non-compliant material which includes compliant material therein.

23. The system of claim 8, wherein the sensing element further includes a supporting member for supporting the moving member and the sensing member, and the supporting member includes a wall portion and an extending base portion, and a ceramic chip substrate secured to the extending base portion, the sensing member comprises a piezo-resistive pressure sensor, and the inner portion of the moving member is adapted to be formed in the wall portion, on the ceramic chip substrate, and over and about the piezo-resistive pressure sensor.

24. The system of claim 8, wherein the moving member is adapted to be formed such that the ratio of the outer portion of the moving member to the interface between the outer portion and the inner portion of the moving member is substantially minimal.

25. The system of claim 10, wherein the supporting member comprises a gel cup, comprised of generally molded plastic material, and filled with a non-conductive silicone gel, a bonding element for bonding the sensing member thereto, and a ceramic chip substrate.

26. The system of claim 10, wherein the supporting member comprises a gel cup, comprised of generally molded plastic material, and a ceramic chip substrate.

27. The system of claim 10, wherein the supporting member comprises a ceramic chip substrate.

28. The system of claim 10, wherein the supporting element includes a portion which is generally semi-spherical in shape, which includes a recess from which the moving member is adapted to project and to be moveable therein, and a generally rectangular-plate-shaped portion.

29. The system of claim 10, wherein the moving member and the supporting member are further adapted to support the sensing member so as to isolate the sensing member from the external environment, to prevent current from flowing to the patient.

30. The system of claim 10, wherein the supporting member includes a plurality of contact pads for receiving a plurality of connectors in a cable from a fetal monitor, further comprising a plurality of wires for connecting the sensing member to the contact pads, and wherein, in the event of the application of a voltage or current exceeding a limit, the wires connecting the sensing member to the contact pads will fail substantially instantaneously, preventing current from flowing to the patient.

31. The system of claim 10, wherein the sensing member and the supporting member comprise a pressure transducer.

32. The system of claim 11, wherein the supporting member is adapted to be formed as a supporting portion of the supporting element.

33. The system of claim 11, wherein the supporting member is adapted to be formed as a supporting part which is adapted to extend between the sensing element and the supporting element.

34. The system of claim 13, wherein the resistors comprise a balanced resistor network.

35. The system of claim 15, wherein the ratio comprises the ratio of the width of the moving member to the width of the sensing member.

36. The system of claim 15, wherein the ratio comprises the ratio of the area of the moving member to the area of the sensing member.

37. The system of claim 17, wherein the flexible material comprises a flexible plastic material.

38. The system of claim 19, wherein the belt is comprised of generally elastic material.

39. The system of claim 21, wherein the generally compliant material comprises a generally compliant polymer material.

40. The system of claim 21, wherein the generally compliant material comprises a generally compliant monomer material.

41. The system of claim 21, wherein the generally compliant material comprises a generally low durometer compliant material.

42. The system of claim 22, wherein the generally non-compliant material comprises a generally non-low durometer non-compliant material.

43. The system of claim 23, wherein the ceramic chip substrate includes compensating resistors.

44. The system of claim 24, wherein the ratio comprises the ratio of the cross-sectional area of the width of the outer portion of the moving member to the cross-sectional area of the interface between the outer portion and the inner portion of the moving member.

45. The system of claim 24, wherein the ratio comprises the ratio of the width of the linear width of the outer portion of the moving member to the linear width of the interface between the outer portion and the inner portion of the moving member.

46. The system of claim 25, wherein the moving member is formed of a material which is adapted to be dispensed and formed relative to the gel cup, the gel cup includes an adhering portion, adapted to enable the moving member to adhere thereto, and the supporting element includes an extending portion, which is formed relative to the adhering portion, and which is adapted to inhibit the material of which the moving member is formed from flowing beyond the adhering portion.

47. The system of claim 25, wherein the ceramic chip substrate includes compensating resistors.

48. The system of claim 26, wherein the ceramic chip substrate includes compensating resistors.

49. The system of claim 27, wherein the ceramic chip substrate includes compensating resistors.

50. The system of claim 32, wherein the sensing member and the supporting portion of the supporting element comprise a pressure transducer.

51. The system of claim 32, wherein the sensing member and the supporting part of the supporting element comprise a pressure transducer.

52. The system of claim 34, wherein the balanced resistor network comprises a silicon semiconductor Wheatstone bridge.

53. The system of claim 35, wherein the width of the moving member and the width of the sensing member comprises the width in any direction thereof.

54. The system of claim 35, wherein the ratio is no greater than about three and one-half to one.

55. The system of claim 36, wherein the ratio is no greater than about eight to one.

56. The system of claim 38, wherein the generally elastic material comprises an elastic nylon material.

57. The system of claim 38, wherein the generally elastic material comprises an elastic polyester material.

58. The system of claim 43, wherein the cross-sectional area of the width of the outer portion of the moving members comprises the cross-sectional area of the widest width thereof.

59. The system of claim 44, wherein the ratio is no greater than about eight to one.

60. The system of claim 45, wherein the width of the linear width of the outer portion of the moving member comprises the width of the linear widest width thereof.

61. The system of claim 45, wherein the ratio is no greater than about three and one-half to one.

62. The system of claim 54, wherein the ratio is about two and one-half to one.

63. The system of claim 55, wherein the ratio is about five to one.

64. The system of claim 59, wherein the ratio is about five to one.

65. The system of claim 61, wherein the ratio is about two and one-half to one.

66. A system for sensing the frequency and duration of uterine contractions during labor and estimating the relative intensity thereof, adapted to sense changes in pressure in response to uterine muscle tone changes relating to contractions, and to enable a fetal monitor to be connected thereto for evaluation of contractions, comprising:
a sensing element, for sensing changes in pressure responsive to uterine muscle tone changes, adapted to be positioned against the exterior abdominal wall proximate the uterine muscle, and to be connected to a fetal monitor for evaluation of contractions during labor;
a supporting element, for supporting the sensing element so as to bear against the exterior abdominal wall proximate the uterine muscle; and
an extending element, for extending about the exterior abdominal wall, adapted to retain the supporting element and the sensing element in position against the exterior abdominal wall proximate the uterine muscle.

67. The system of claim 66, wherein the sensing element includes a moving member, for moving by displacing or compressing so as to exert pressure responsive to uterine muscle tone changes, a sensing member, for sensing movement of the moving member, and for enabling a fetal monitor to be connected thereto, and a supporting member, for supporting the moving member and the sensing member.

68. The system of claim 66, wherein the supporting element is adapted to support the sensing element such that the sensing element projects into the abdominal wall so as to reside below the non-compressed surface of the abdomen.

69. The system of claim 66, wherein the supporting element is adapted to leverage the sensing element, so as to enable the extending element to load the sensing element onto the abdomen, with substantially minimal retention pressure exerted by the extending element on the sensing element.

70. The system of claim 66, wherein the supporting element is comprised of a flexible material, such that, in conjunction with the extending element, the flexible material maintains a load on the sensing element during abdominal changes relating to contractions.

71. The system of claim 67, wherein the moving member and the supporting member are further adapted to support the sensing member so as to isolate the sensing member from the external environment, to prevent current from flowing to the patient.

72. A method of sensing the frequency and duration of uterine contractions during labor and estimating the relative intensity thereof, adapted to sense changes in pressure responsive to uterine muscle tone changes relating to contractions, and to enable a fetal monitor to be connected thereto for evaluation of contractions, in a system which comprises a sensing element, for sensing changes in pressure responsive to uterine muscle tone changes, adapted to be positioned against the exterior abdominal wall proximate the uterine muscle, and to be connected to a fetal monitor for evaluation of contractions during labor, and a supporting element, for supporting the sensing element so as to bear against the exterior abdominal wall proximate the uterine muscle, wherein the method comprises:
supporting the sensing element so as to be positionable to bear against the exterior abdominal wall proximate the uterine muscle, in the supporting element; and
sensing changes in pressure responsive to uterine muscle tone changes, in the sensing element.

73. The method of claim 72, wherein the sensing element includes a moving member, for moving by displacing or compressing responsive to uterine muscle tone changes, and a sensing member, for sensing movement of the moving member, and for enabling a fetal monitor to be connected thereto, and wherein sensing includes moving the moving member responsive to uterine muscle tone changes, and sensing movement of the moving member.

74. The method of claim 72, wherein the sensing element is generally small and lightweight, and the supporting element is generally low profile and lightweight, and wherein sensing further comprises sensing pressure changes, in the generally small and lightweight sensing element.

75. The method of claim 72, wherein the sensing element is further adapted to be actuated by an excitation voltage from a fetal monitor, and to send a signal voltage back to the fetal monitor responsive to changes in pressure, further comprising actuating the sensing element with an excitation voltage from a fetal monitor, and sending a signal back to the fetal monitor responsive to pressure changes.

76. The method of claim 72, further comprising an extending element, for extending about the exterior abdominal wall, adapted to retain the supporting element and the sensing element in position against the exterior abdominal wall proximate the uterine muscle, and further comprising retaining the supporting element and the sensing element in position against the exterior abdominal wall proximate the uterine muscle, in the extending element.

77. The method of claim 72, wherein the supporting element is adapted to support the sensing element such that the sensing element projects into the abdominal wall so as to reside below the non-compressed surface of the abdomen, and wherein supporting further comprises supporting the sensing element such that the sensing element projects into the abdominal wall so as to reside below the non-compressed surface of the abdomen.

78. The method of claim 72, further comprising connecting the sensing element to a fetal monitor for evaluation of contractions during labor.

79. The method of claim 73, wherein the moving member comprises a force collector, and wherein moving comprises moving the force collector.

80. The method of claim 73, wherein the moving member includes an outer portion adapted to project from the supporting member, an inner portion adapted to contact the sensing member, and an interface between the outer portion and the inner portion, and wherein moving further comprises moving the generally soft and compliant moving member, including projecting the outer portion thereof, and contacting the inner portion thereof with the sensing member.

81. The system of claim 73, wherein the moving member is comprised of material which is generally soft and compliant, and wherein sensing further includes moving the generally soft and compliant moving member responsive to uterine muscle tone changes, and sensing movement of the generally soft and compliant moving member.

82. The method of claim 73, wherein the sensing element further includes a supporting member, for supporting the moving member and the sensing member, and wherein supporting further comprises supporting the moving member and the sensing member in the supporting member.

83. The method of claim 73, wherein the supporting element includes a supporting member for supporting the moving member, and wherein supporting further comprises supporting the moving member and the sensing member in the supporting member.

84. The method of claim 73, wherein the moving member and the supporting member are further adapted to support the sensing member so as to isolate the sensing member from the external environment, to prevent current from flowing to the patient, and wherein supporting further comprises supporting the sensing element in the moving member and the supporting member so as to isolate the sensing element from the external environment.

85. The method of claim 73, wherein the sensing member comprises the active area of the sensing element, and includes resistors adapted to change the resistance with changes in applied pressure, and wherein sensing further comprises changing the resistance of the resistors in the sensing member with changes in applied pressure.

86. The method of claim 73, wherein the supporting element is adapted to support the moving member such that the moving member projects into the abdominal wall so as to reside below the non-compressed surface of the abdomen, and wherein supporting further comprises supporting the moving member such that the moving member projects into the abdominal wall so as to reside below the non-compressed surface of the abdomen.

87. The method of claim 73, wherein the moving member is adapted to be formed such that the ratio of the moving member to the sensing member is substantially minimal, further comprising forming the moving member such that the ratio of the moving member to the sensing member is substantially minimal.

88. The method of claim 76, wherein the supporting element is adapted to leverage the sensing element, so as to enable the extending element to load the sensing element onto the abdomen, with substantially minimal retention pressure exerted by the extending element on the sensing element, and wherein supporting further comprises leveraging the sensing element in the supporting element.

89. The method of claim 76, wherein the supporting element is comprised of a flexible material, such that, in conjunction with the extending element, the flexible material maintains a load on the sensing element during abdominal changes relating to contractions, and wherein supporting further comprises maintaining a load on the sensing element in the supporting element and the extending element.

90. The method of claim 76, wherein the supporting element is comprised of a non-flexible material, such that, in conjunction with the extending element, the non-flexible material maintains a load on the sensing element during abdominal changes relating to contractions, and wherein supporting further comprises maintaining a load on the sensing element in the supporting element and the extending element.

91. The method of claim 76, wherein the extending element comprises a belt, and wherein retaining further comprises retaining the sensing element in position in the belt.

92. The method of claim 77, wherein the supporting element includes a housing which includes a back plate, and a dome, projecting from the back plate, and wherein the dome has a well therein, and the sensing element is adapted to be mounted in the well of the dome, and wherein supporting further comprises supporting the sensing member in the well of the dome of the supporting element housing.

93. The method of claim 79, wherein the force collector is generally in the shape of a nipple, and is comprised of a generally compliant material, and wherein moving further comprises moving the generally nipple-shaped force collector.

94. The method of claim 79, wherein the force collector is generally in the shape of a nipple, and is comprised of a generally non-compliant material which includes compliant material therein, and wherein moving further comprises moving the generally nipple-shaped force collector.

95. The method of claim 79, wherein the sensing element further includes a supporting member for supporting the moving member and the sensing member, and the supporting member includes a wall portion and an extending base portion, and a ceramic chip substrate secured to the extending base portion, the sensing member comprises a piezo-resistive pressure sensor, and the inner portion of the moving member is adapted to be formed in the wall portion, on the ceramic chip substrate, and over and about the piezo-resistive pressure sensor, further comprising forming the inner portion of the moving member in the wall portion and on the ceramic chip substrate of the supporting member, and over and about the piezo-resistive pressure sensor of the sensing member.

96. The method of claim 80, wherein the moving member is adapted to be formed such that the ratio of the outer portion of the moving member to the interface between the outer portion and the inner portion of the moving member is substantially minimal, further comprising forming the moving member such that the ratio of the outer portion of the moving member to the interface between the outer portion and the inner portion of the moving member is substantially minimal.

97. The method of claim 82, wherein the supporting member comprises a gel cup, comprised of generally molded plastic material, and filled with a non-conductive silicone gel, a bonding element for bonding the gel cup to the sensing member thereto, and a ceramic chip substrate, further comprising supporting the moving member in the gel cup of the supporting member.

98. The method of claim 82, wherein the supporting member comprises a gel cup, comprised of generally molded plastic material, and a ceramic chip substrate, further comprising supporting the moving member in the gel cup of the supporting member.

99. The method of claim 82, wherein the supporting member comprises a ceramic chip substrate, further comprising supporting the moving member on the ceramic chip substrate of the supporting member.

100. The method of claim 82, wherein the supporting element includes a portion which is generally semi-spherical in shape, which includes a recess from which the moving member is adapted to project and to be moveable therein, and a generally rectangular-plate-shaped portion, and wherein supporting further comprises supporting the moving member in the recess in the generally semi-spherically shaped portion of the supporting element.

101. The method of claim 82, wherein the moving member and the supporting member are further adapted to support the sensing member so as to isolate the sensing member from the external environment, to prevent current from flowing to the patient, and wherein supporting further comprises supporting the sensing element in the moving member and the supporting member so as to isolate the sensing element from the external environment.

102. The method of claim 82, wherein the supporting member includes a plurality of contact pads for receiving a plurality of connectors in a cable from a fetal monitor, further comprising a plurality of wires for connecting the sensing member to the contact pads, and wherein, in the event of the application of a voltage or current exceeding a limit, the wires connecting the sensing member to the contact pads will fail substantially instantaneously, preventing current from flowing to the patient, further comprising substantially instantaneously causing the wires connecting the sensing member to the fetal monitor cable to fail substantially instantaneously in the event of the application of a voltage or current exceeding the limit.

103. The method of claim 82, wherein the sensing member and the supporting member comprise a pressure transducer, and wherein sensing further comprises sensing movement of the moving member in the pressure transducer.

104. The method of claim 83, wherein the supporting member is adapted to be formed as a supporting portion of the supporting element, and wherein supporting further comprises supporting the moving member and the sensing member in the supporting portion of the supporting member.

105. The system of claim 83, wherein the supporting member is adapted to be formed as a supporting part which is adapted to extend between the sensing element and the supporting element, and wherein supporting further comprises supporting the moving member and the sensing member in the supporting part extending between the sensing element and the supporting member.

106. The method of claim 85, wherein the resistors comprise a balanced resistor network, and wherein sensing further comprises changing the resistance of the balanced resistor network in the sensing member with changes in applied pressure.

107. The method of claim 87, wherein the ratio comprises the ratio of the width of the moving member to the width of the sensing member, and wherein forming further comprises forming the moving member such that the ratio of the width of the moving member to the width of the sensing member is substantially minimal.

108. The method of claim 87, wherein the ratio comprises the ratio of the area of the moving member to the area of the sensing member, and wherein forming further comprises forming the moving member such that the ratio of the area of the moving member to the area of the sensing member is substantially minimal.

109. The method of claim 89, wherein the flexible material comprises a flexible plastic material, and wherein supporting further comprises maintaining a load on the sensing element in the flexible plastic material supporting element and in the extending element.

110. The method of claim 91,wherein the belt is comprised of generally elastic material, and wherein retaining further comprises retaining the sensing element in position in the generally elastic belt.

111. The method of claim 93, wherein the generally compliant material comprises a generally compliant polymer material, and wherein moving further comprises moving the generally nipple-shaped compliant polymer material force collector.

112. The method of claim 93, wherein the generally compliant material comprises a generally compliant monomer material, and wherein moving further comprises moving the generally nipple-shaped compliant monomer material force collector.

113. The method of claim 93, wherein the generally compliant material comprises a generally low durometer compliant material, and wherein moving further comprises moving the generally nipple-shaped low durometer compliant polymer material force collector.

114. The method of claim 94, wherein the generally non-compliant material comprises a generally non-low durometer non-compliant material, and wherein moving further comprises moving the generally nipple-shaped non-low durometer compliant material force collector.

115. The method of claim 95, wherein the ceramic chip substrate includes compensating resistors, and wherein forming further comprises forming the inner portion of the moving member in the wall portion and on the ceramic compensating resistor chip substrate.

116. The method of claim 96, wherein the ratio comprises the ratio of the cross-sectional area of the width of the outer portion of the moving member to the cross-sectional area of the interface between the outer portion and the inner portion of the moving member, and wherein forming further comprises forming the moving member such that the ratio of the cross-sectional area of the width of the outer portion of the moving member to the cross-sectional area of the interface between the outer portion and the inner portion of the moving member is substantially minimal.

117. The method of claim 96, wherein the ratio comprises the ratio of the width of the linear width of the outer portion of the moving member to the linear width of the interface between the outer portion and the inner portion of the moving member, and wherein forming further comprises forming the moving member such that the ratio of the linear width of the outer portion of the moving member to the linear width of the interface between the outer portion and the inner portion of the moving member is substantially minimal.

118. The method of claim 97, wherein the moving member is formed of a material which is adapted to be dispensed and formed relative to the gel cup, the gel cup includes an adhering portion, adapted to enable the moving member to adhere thereto, and the supporting element includes an extending portion, which is formed relative to the adhering portion, and which is adapted to inhibit the material of which the moving member is formed from flowing substantially beyond the adhering portion, and wherein supporting the moving member in the gel cup further comprises adhering the force collector to the adhering portion of the gel cup, and inhibiting the flow of the moving member substantially beyond the adhering portion of the gel cup.

119. The method of claim 97, wherein the ceramic chip substrate includes compensating resistors, and wherein supporting further comprises supporting the moving member on the ceramic compensating resistor chip substrate.

120. The method of claim 98, wherein the ceramic chip substrate includes compensating resistors, and wherein supporting further comprises supporting the moving member on the ceramic compensating resistor chip substrate.

121. The method of claim 99, wherein the ceramic chip substrate includes compensating resistors, and wherein supporting further comprises supporting the moving member on the ceramic compensating resistor chip substrate.

122. The method of claim 104, wherein the sensing member and the supporting portion of the supporting element comprise a pressure transducer, and wherein sensing further comprises sensing movement of the moving member in the pressure transducer.

123. The method of claim 105, wherein the sensing member and the supporting part of the supporting element comprise a pressure transducer, and wherein sensing further comprises sensing movement of the moving member in the pressure transducer.

124. The method of claim 105, further comprising forming the moving member by dispensing the material of which it is comprised from a syringe in drops, and rapidly curing the material with ultraviolet light.

125. The method of claim 106, wherein the balanced resistor network comprises a silicon semi-conductor Wheatstone bridge, and wherein sensing further comprises sensing movement in the silicon semi-conductor Wheatstone bridge of the balanced resistor network.

126. The method of claim 107, wherein the width of the moving member and the width of the sensing member comprises the width in any direction thereof, and wherein forming further comprises forming the moving member such that the ratio of the width in any direction of the moving member to the width in any direction of the sensing member is substantially minimal.

127. The method of claim 107, wherein the ratio is no greater than about three and one-half to one, and wherein forming further comprises forming the moving member such that the ratio is no greater than about three and one-half to one.

128. The method of claim 108, wherein the ratio is no greater than about eight to one, and wherein forming further comprises forming the moving member such that the ratio is no greater than about eight to one.

129. The method of claim 110, wherein the generally elastic material comprises an elastic nylon material, and wherein retaining further comprises retaining the sensing element in position in the generally elastic nylon belt.

130. The method of claim 110, wherein the generally elastic material comprises an elastic polyester material, and wherein retaining further comprises retaining the sensing element in position in the generally elastic polyester belt.

131. The method of claim 116, wherein the cross-sectional area of the width of the outer portion of the moving member comprises the cross-sectional area of the widest width thereof, and wherein forming further comprises forming the moving member such that the ratio of the cross-sectional area of the widest width of the outer portion of the moving member to the cross-sectional area of the interface between the outer portion and the inner portion of the moving member is substantially minimal.

132. The method of claim 116, wherein the ratio is no greater than about eight to one, and wherein forming further comprises forming the moving member such that the ratio is no greater than about eight to one.

133. The method of claim 117, wherein the width of the linear width of the outer portion of the moving member comprises the width of the linear widest width thereof, and wherein forming further comprises forming the moving member such that the ratio of the linear widest width of the outer portion of the moving member to the linear width of the interface between the outer portion and the inner portion of the moving member is substantially minimal.

134. The method of claim 117, wherein the ratio is no greater than about three and one-half to one, and wherein forming further comprises forming the moving member such that the ratio is no greater than about three and one-half to one.

135. The method of claim 127, wherein the ratio is about two and one-half to one, and wherein forming further comprises forming the moving member such that the ratio is about two and one-half to one.

136. The method of claim 132, wherein the ratio is about five to one, and wherein forming further comprises forming the moving member such that the ratio is about five to one.

137. The method of claim 132, wherein the ratio is about five to one, and wherein forming further comprises forming the moving member such that the ratio is about five to one.

138. The method of claim 134, wherein the ratio is about two and one-half to one, and wherein forming further comprises forming the moving member such that the ratio is no greater than about two and one-half to one.
